(19) United States
(12) Patent Application Publication
(10) Pub. No.: US 2009/0226600 A1
(43) Pub. Date: Sep. 10, 2009

(54) Surface Coating Method and Coated Device

(76) Inventors: Mai Huong Dang, Palo Alto, CA (US); Phillip Chiu, San Francisco, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/466,626

(22) Filed: May 15, 2009

Related U.S. Application Data
(63) Continuation of application No. 10/017,193, filed on Dec. 12, 2001.

(51) Int. Cl. A61L 27/54

(52) U.S. Cl. 427/2.25

(57) ABSTRACT

A multi-step method of forming a coating on a substrate, such as a stent or graft, is disclosed. The steps of the method include treating the surface with a plasma formed at or near atmospheric pressure to form one or more active species on the surface until a desired surface density of the active species is formed, and exposing the treated surface to a selected gas or liquid under conditions effective to convert the active species to a stable functional group. The exposed surface may be contacted with a surface-modifying group under conditions effective to covalently attach the surface-modifying group to the functional group. Also disclosed is a substrate having a bioactive/biocompatible coating and/or a drug-releasable coating prepared by the method.

### CROSS-RELATED APPLICATIONS

This application is a continuation of U.S. patent application Ser. No. 10/017,193, filed Dec. 20, 2001, and is incorporated herein in its entirety.

### FIELD OF THE INVENTION

The present invention relates to a method for forming a coating on the surface of a substrate, and to devices useful in practicing the method.

### REFERENCES

Alberts, B. et al. (1994), Molecular Biology of the Cell, 3rd ed., Garland Publ., Inc., New York.
Bhatnager, R. S. et al. (1997), The Role in Cell Binding of a β-bend within the Triple Helical Region in Collagen α1(I) Chain: Structural and Biological Evidence for Conformational Tautomerism on Fiber Surface. J Biomolec Stuc & Dynam 14(5):547-560.
Bhatnager, R. S. et al. (1999), Design of Biomimetic Habitats for Tissue Engineering with P-15, a Synthetic Peptide Analog of Collagen. Tissue Engineering 5(1):53-65.
Brinkley, M. (1992), A Brief Survey Of Methods For Preparing Protein Conjugates With Dyes, Haptens, and Cross-linking Reagents, Bioconjugate Chem., 3:2.
Charonis, A. S., et al. (1988), J. Cell Biol. 107:1253-1260.
Hermanson, G. T., et al. (992) Immobilized Affinity Ligand Techniques, Academic Press, San Diego, Calif.
Horton, H. R. and Swaisgood, H. E., (1987) Covalent immobilization of proteins by techniques which permit subsequent release, Meth. Enzymology, 135: 130.
Hubbell, J. A., et al. (1992), Ann. N.Y. Acad. Sci. 665:253-258.
Kanazawa, T., et al. (1995), Development of a Hydrophilic PTFE porous membrane filter. Sumitomo Denki, 147:99.
Kleinman, H. K, et al. (1993), Vitamins and Hormones 47:161-186.
Koliakos, G. G., et al. (1989), J. Biol. Chem. 264:2313-2323.
Manners, I. (2001), Putting metals into Polymers, Science 294:1664-1666.
Mooradian, D. L., et al. (1993) Invest. Ophth. & Vis. Sci. 34:153-164.
Mosbach, K., (1987) Immobilized Enzymes and Cells, Part B, Academic Press, Orlando, Fla.
Pointer, A. B. et al., (1994) Surface Energy Changes Produced by Ultraviolet-ozone Irradiation of Poly(methyl methacrylate), Polycarbonate, and Polytetrafluoroethylene. Polym. Eng. Sci., 34:1233.
Schneider, D B and Dichek D A (1997), Intravascular Stent Endotheliazation. A Goal Worth Pursuing? Circulation 95:308-10.
Schwartz, R. S. (1997), The Vessel Wall Reaction in Restenosis. Semin Intervent Cardiol 2:83-8.
Wong, S. S. (1991) Chemistry of Protein Conjugation and Cross-Linking, CRC Press.
Yutani, C. et al. (1999), Coronary Atherosclerosis and Interventions: Pathological Sequences and Restenosis. Path Int 49:273-90.
Zazloff, M. (1992), Curr. Opinion Immunol. 4:3-7.

### BACKGROUND OF THE INVENTION

Endovascular devices such as stents, stent grafts and vascular grafts are widely used to treat de novo and restenotic vascular lesions. Although these devices have improved the results over angioplasy alone, failure rates remain high (Yutani et al., 1999; Schwarz, 1997). This is especially true in small diameter vessels and saphenous vein grafts. Although short-term failure (1-2 weeks), mostly caused by a thrombotic response to the devices, can be managed with medications, long-term restenosis (3-6 months), resulting from a complex cascade of injury, inflammation response and intimal hyperplasia, continues to be a challenge for small-diameter vascular devices.

Regeneration of a functional endothelium over the surfaces of the implanted devices promises to be a long-term solution for the reduction and prevention of thrombosis and intimal hyperplasia. (Schneider and Dichek, 1997). Devices having biomimetic surfaces coated with sequences of extracellular matrix proteins, such as collagen or laminin, could improve and accelerate endothelial regeneration. However, modifying or coating the surfaces of such devices, such as ePTFE grafts, has been difficult and often unsuccessful (EP-B 910584). Due to the extreme chemical inertness of the backbone of fluorocarbon polymers and many hydrocarbon polymers, highly energetic classes of reactions have been used to alter the backbone of these materials to produce chemically reactive organic moieties thereon. Previously described coating methods utilizing these reactions have been less than optimal (U.S. Pat. No. 5,462,781; Pointer, et al., 1994; and Kanazawa, et al., 1995), and many require maintaining the substrate in a vacuum during the treatment process.

The use of atmospheric plasma as a coating process provides a number of advantages over vacuum plasma processes. These include: (1) the low cost for equipment, installation, operation and maintenance—for example, there is no need for tightly sealed vacuum systems (pumps, traps and cooling to prevent oil vapor backlash, sealing, etc.); (2) the ability to perform high throughput, highly automated processes using conventional manufacturing equipment such as conveyors; and (3) the ability to treat the inside of long, narrow, cylindrical substrates by directing atmospheric plasma flow to the surface of the substrate without a significant increase in the temperature of the substrate. In contrast, the inside of narrow tubes cannot be treated in a conventional vacuum plasma chamber because plasma glow is neither generated nor able to diffuse easily into the narrow tubes by passive diffusion. If the glow is forced to enter tubes, its energy almost immediately heats up, deforms and melts plastic tubes. Furthermore, it is easier and cheaper to add additional gases or vapors into the plasma flow, therefore varying the treatment for different materials, in the atmospheric plasma process.

It would therefore be valuable to provide a surface coating method that does not require treatment inside a vacuum chamber, and is capable of achieving a desired surface density of a selected chemical group on a substrate. Medical devices treated in this fashion would be highly effective in a physiological environment. The present invention is designed to meet these needs.

### SUMMARY OF THE INVENTION

The invention includes, in one embodiment, a method of forming on the surface of a substrate a coating having a selected surface density of a selected chemical group. The method includes the steps of treating the surface with a plasma formed at or near atmospheric pressure to form one or more active species on the surface; continuing the treating until a desired surface density of the active species is formed; and exposing the treated surface to a selected gas or liquid under conditions effective to convert the active species to a stable functional group. The exposed surface may be contacted with a surface-modifying group or linker under conditions effective to covalently attach the surface-modifying group to said functional group. Thus, the selected chemical group on the surface is the stable functional group or the surface-modifying group covalently attached thereto. The surface-modifying group or linker may further react with a biological active component resulting in a substrate with a bioactive surface or in a substrate which provides a local and sustained release of a bioactive component.

Treating may be performed by streaming the plasma through or against the surface of the substrate or maintaining the substrate in a space enriched with active plasma species at or near atmospheric pressure. The substrate may have a tubular shape and the plasma may flow between the outside surface of the substrate and the inside surface of a surrounding substrate to confine and extend the plasma.

In one embodiment, the surface is a non-porous or porous polymer. A preferred surface is porous expanded PTFE.

In another embodiment, the plasma is formed of a carrier gas and less than ten percent of a gas or vapor selected from the group consisting of oxygen, water, ammonia, ammonium hydroxide, an organic amine, an alcohol, an aldehyde, a carboxylic acid and an ester. In yet another embodiment, the plasma is formed of an carrier gas and greater than 0.1 percent of a gas or vapor selected from the group consisting of oxygen, water, ammonia, ammonium hydroxide, an organic amine, an alcohol, an aldehyde, a carboxylic acid, and an ester. In a related embodiment, a reactive gas in an amount sufficient to extinguish the plasma glow is added.

In yet another embodiment, the substrate is a conductive metal capable of being a first electrode, and the treating includes forming a plasma around and in contact with the metal surface and a second, plasma-generating electrode adjacent thereto. The metal substrate is preferably a cylinder. In this embodiment, the second plasma-generating electrode may be positioned near the substrate. Alternatively, the metal substrate is a cylinder defining a bore extending therethrough along a longitudinal axis, and the second, plasma-generating electrode is positioned through said bore along said longitudinal axis.

The exposing step may be performed by contacting the surface with a substance selected from the group consisting of air, ammonia, oxygen, all in gaseous form, and water, ammonium hydroxide, and hydrazine, all in liquid form.

The surface-modifying group may be a multifunctional linker selected from the group consisting of anhydrides, alcohols, acids, amines, epoxies, isocyanates, silanes, halogenated groups, and polymerizable groups. The multifunctional linkers may be selected from the group consisting of halogenated carboxylic acid. The halogenated carboxylic acid may be selected from the group consisting of chloroacetic acid, chlorobutyric acid, and chlorovaleric acid. The multifunctional linker is preferably comprised of at least one molecule with 2-20 carbon atoms in the backbone. In one embodiment, the multifunctional linker is a string formed of heterofunctional molecules. Alternatively, the multifunctional linker is a string formed of alternate homofunctional molecules.

In another embodiment, the surface-modifying group may be a more reactive group formed between the plasma activated surface and activating agents such as tosyl chloride, tresyl chloride or mesyl chloride, which facilitates the efficient covalent attachment of the bioactive/biocompatible coating under mild conditions.

In yet another embodiment, the invention includes a substrate having a coating with a selected surface density of a selected chemical group prepared by a process that includes the steps of treating the surface with a plasma formed at or near atmospheric pressure to form one or more active species on said surface; continuing said treating until a desired surface density of the active species is formed; exposing the treated surface to a selected gas or liquid under conditions effective to convert the active species to a stable functional group; and optionally contacting the exposed surface to a surface-modifying group under conditions effective to covalently attach the surface-modifying group to said functional group, where the selected chemical group on the surface is the stable functional group or the surface-modifying group covalently attached thereto.

In another aspect, the surface may be further contacted with a bioactive or biocompatible agent to form a covalent or non-covalent bond between the bioactive or biocompatible agent and the stable functional group 16 or the surface modifying group 18 to bind the bioactive or biocompatible agent to the substrate surface. The bioactive or biocompatible agent may be selected from the group consisting of a protein, a peptide, an amino acid, a carbohydrate, and a nucleic acid, each being capable of binding covalently or noncovalently to specific and complementary portions of molecules or cells. The bioactive or biocompatible agent may also be selected from the group consisting a cell attachment factor, a receptor, a ligand, a growth factor, of an antithrombotic agent, an antibiotic, and an enzyme.

The cell attachment factor may be selected from the group consisting of a surface adhesion molecule and a cell-cell adhesion molecule. The surface adhesion molecule may be selected from the group consisting of laminin, fibronectin, collagen, vitronectin, tenascin, fibrinogen, thrombospondin, osteopontin, von Willibrand Factor, and bone sialoprotein, and active domains thereof. In one embodiment, the cell-cell adhesion molecule is P15, a cell adhesion domain of collagen I (SEQ ID NO: 1). The cell-cell adhesion molecule may be selected from the group consisting of N-cadherin and P-cadherin and active domains thereof.

The growth factor may be selected from the group consisting of a fibroblastic growth factor, an epidermal growth factor, a platelet-derived growth factor, a transforming growth factor, a vascular endothelial growth factor, a bone morphogenic protein, and a neural growth factor. The ligand or receptor may be selected from the group consisting of an antibody, an antigen, avidin, streptavidin, biotin, heparin, type IV collagen, protein A, and protein G. In one embodiment, the antibiotic is an antibiotic peptide. The bioactive or biocompatible agent may be an enzyme. Alternatively, the bioactive or biocompatible agent includes a nucleic acid sequence capable of selectively binding complementary sequences.

The antithrombotic agent may be selected from the group consisting of heparin, hirudin, lysin such as plasmin fibrinolysin or thrombolysin, plasminogen activator, prostaglandin, streptokinase, and urokinase.

In one embodiment, the bioactive or biocompatible agent is covalently attached to the surface at a density of 0.01 to 1000 nmol/cm². Preferably, the bioactive or biocompatible agent is bound to the surface at a density of 0.5 to 30 nmol/cm². More preferably, the bioactive or biocompatible agent is bound to the surface at a density of 2 to 20 nmol/cm². These and other objects and features of the invention will be more fully appreciated when the following detailed description of the invention is read in conjunction with the accompanying drawings.

### BRIEF DESCRIPTION OF THE DRAWINGS

FIG. 1 is a flow chart showing the method of the present invention;

FIG. 2 shows the treatment of the inside surface of a cylindrical substrate according to one embodiment of the invention;

FIG. 3 shows the treatment of a surface of a flat substrate according to another embodiment of the invention;

FIG. 4 shows the treatment of the outside surface of a cylindrical substrate according to yet another embodiment of the invention;

FIG. 5 illustrates the change in P-15 concentration as a function of wetting agent composition;

FIG. 6 illustrates the change in P-15 concentration as a function of plasma activation conditions and composition;

FIGS. 7A-7B illustrate 20× magnifications of the migration of endothelial cells on control (7A) and treated (7B) ePTFE grafts following 13 days of culture;

FIGS. 8A-8B illustrate 500× magnifications of the migration of endothelial cells on control (8A) and treated (8B) ePTFE grafts following 13 days of culture.

### DETAILED DESCRIPTION OF THE INVENTION

### I. Definitions

Unless otherwise indicated, all technical and scientific terms used herein have the same meaning as they would to one skilled in the art of the present invention. It is to be understood that this invention is not limited to the particular methodology, protocols, and reagents described, as these may vary.

As used herein, the terms “implant” and “implanted” include devices and substrates that are implanted into the body, e.g. stents and grafts, as well as to devices that are applied to the skin surface of a patient, e.g., a wound dressing.

As used herein, the term “immobilize,” and its derivatives, refers to the attachment of a bioactive species directly to a substrate, or to a substrate through at least one intermediate component.

As used herein, the terms “bioactive” or “biocompatible” or “bioactive/biocompatible” will refer to a molecule having a desired specific biological activity, such as a binding or enzymatic activity or to a surface with desired biological properties such as non-thrombogenic surface.

All publications and patents cited herein are expressly incorporated herein by reference for the purpose of describing and disclosing compositions and methodologies that might be used in connection with the invention.

### II. Method of the Invention

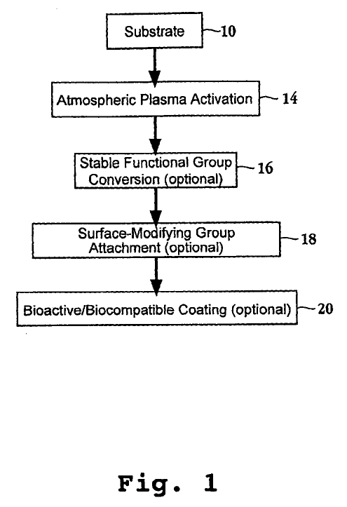

The invention includes, in one aspect, a method of forming a coating on the surface of a substrate. It has been discovered that a coating having a selected surface density of a selected chemical group has a number of beneficial biological uses. Considered below are the steps as illustrated in FIG. 1 in practicing the invention.

A. Substrate

Referring to FIG. 1, the method of the invention employs a substrate 10 onto which a coating is formed. The nature of the substrate to be coated may vary widely. At least a portion of at least one surface of the substrate 10 is coated with the functional group 16 or surface-modifying group 18 of the present invention. Preferably, the entire surface is coated with the functional group 16 or surface-modifying group 18. Suitable substrate materials include all non-porous or porous polymeric substrates, such as polyurethanes, polyamides, polyesters and polyethers, polyether-blockamides, polystyrene, polyvinyl chloride, polycarbonates, polyorganosiloxanes, polyolefins, polysulfones, polyisoprene, polychloroprene, polytetrafluorothylene (PTFE), polysiloxanes, fluorinated ethylene propylene, hexafluroropropylene, polyethylene, polypropylene, nylon, polyethyleneterephthalate, polyurethane, silicone rubber, polysulfone, polyhydroxyacids, polyimide, polyamide, polyamino acids, regenerated cellulose, corresponding copolymers and blends, and also natural and synthetic rubbers. A substrate of particular interest to the present invention is expanded PTFE (ePTFE). Methods of making expanded polytetrafluoroethylene materials are described in U.S. Pat. Nos. 3,953,566 and 4,187,390, each of which is incorporated herein by reference.

The method according to the invention can also be applied to metals, ceramics, and metal-polymer composites (Manners, 2001), as well as surfaces of painted or otherwise polymer-coated glass or wooden structures. Suitable metals for support members include, but are not limited to, titanium, stainless steel, gold, silver, rhodium, zinc, platinum, rubidium, and copper, for example. Suitable materials for ceramic support members include, but are not limited to, silicone oxides, aluminum oxides, alumina, silica, hydroxyapapitites, glasses, calcium oxides, polysilanols, and phosphorous oxide, for example. The surfaces of the substrate materials are advantageously freed from adhering oils, greases and other contaminants in a known manner before the coating process.

B. Substrate Activation

The first step of the method involves treating the surface of the substrate 10 with a plasma formed at or near atmospheric pressure as illustrated in step 14 in FIG. 1. It will be understood that “at or near atmospheric pressure” means any pressure above a vacuum pressure of about 10% atmospheric pressure, e.g. 76 torr, and below a hyperbaric pressure of about 2 times atmospheric pressure. Preferably “at or near atmospheric pressure” refers to the pressure within a chamber which is open to the atmosphere, the actual pressure of which will depend on elevation and atmospheric pressure conditions. A preferred pressure is between 700 and 800 torr.

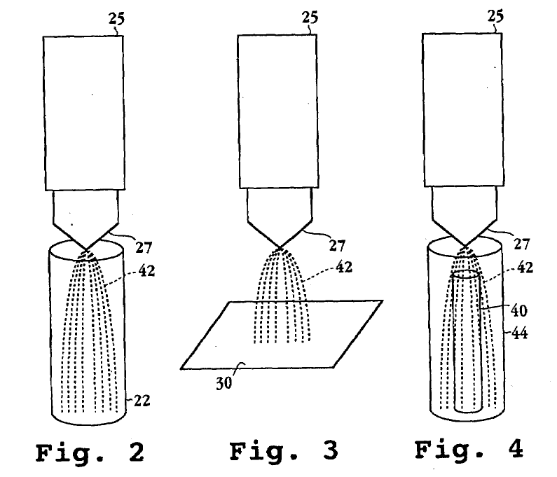

As shown in FIGS. 2-4, the treating step may be performed by streaming the plasma through or against the surface of the substrate 22, 30, or 40. Such treatments may be performed in either an open or confined space. For example, as illustrated in FIG. 4, in order to treat the outside of a tubular-shaped substrate 40, the plasma 42 may flow between the outside surface of the substrate 40 and the inside surface of a surrounding substrate 44. In this manner, the plasma 42 is confined and/or extended.

The plasma treatment process parameters may vary depending on desirable concentrations of functional groups and on the mechanical properties of the substrate. The length of treatment time and volume per minute of plasma passing through the plasma chamber may be varied accordingly. For example, if the substrate is ePTFE, a longer plasma treatment time may increase the density of the active species formed, but may weaken the substrate to a level below the acceptable range. The voltage for plasma treatment may vary according to the equipment used and is readily determined by one of skill in the art. An exemplary set of parameters for treatment of an 8 cm ePTFE cylinder is 12 V, with 30 scfm plasma passing through or against the substrate for 30 seconds.

Referring again to FIGS. 2-4, the invention provides a plasma chamber 25 having a plasma nozzle 27 for forming plasma 42. Any atmospheric plasma generator known to those of skill in the art may be used in the present invention. An exemplary plasma generator is described in U.S. Pat. No. 5,798,146, which is incorporated by reference herein.

The plasma used to achieve activation of the substrate may be formed of an carrier gas alone, or an carrier gas and less than ten percent of a gas or vapor such as oxygen, water, ammonia, ammonium hydroxide, an organic amine, an alcohol, an aldehyde, a carboxylic acid or an ester. In one embodiment, the plasma is formed of a carrier gas and greater than 0.1 percent of a gas or vapor selected from the group consisting of oxygen, water, ammonia, ammonium hydroxide, an organic amine, an alcohol, an aldehyde, a carboxylic acid, and an ester. In one embodiment, the plasma is formed of a carrier gas and between 0.1 and ten percent of oxygen, water, ammonia, ammonium hydroxide, an organic amine, an alcohol, an aldehyde, a carboxylic acid, and/or an ester. A carrier gas may be selected for its ability to sustain plasma glow in the gas flow. Exemplary carrier gasses include nitrogen, carbon dioxide, and noble gasses, e.g. argon, krypton, neon, xenon, and helium. A preferred carrier gas is argon. In this, manner one or more active species are formed on the surface. The treating is continued until a desired surface density of the active species is formed.

The substrate activation step 14 may be performed as a continuous or pulsed plasma process as described in U.S. Pat. No. 6,159,531 which is incorporated by reference in its entirety herein. The power to generate the plasma may be supplied in pulsed form or may be supplied in a graduated or gradient manner, with higher power being supplied initially, followed by the power being reduced or tapered towards the end of the plasma deposition process.

Plasma-assisted activation is preferred because it is a clean, solvent-free process which can activate almost all substrates including the chemically most stable fluoropolymers such as expanded PTFE (ePTFE). Plasma produces high energy species, i.e. ions or radicals, from precursor gas molecules. These high energy species activate the substrate 10 enabling stable bonding between the substrate 10 and surface-modifying groups 18 and/or bioactive/biocompatible coatings 20.

In one embodiment, the substrate is a conductive material, such as metal, capable of being a first electrode, and the treating includes forming a plasma around and in contact with the metal surface and a second, plasma-generating electrode adjacent thereto. The metal substrate is preferably a cylinder. In this embodiment, the second plasma-generating electrode may be positioned near the substrate. Alternatively, the metal substrate is a cylinder defining a bore extending therethrough along a longitudinal axis, and the second, plasma-generating electrode is positioned through the bore along the longitudinal axis. The substrate and second electrode may be either confined within a chamber, or be unconfined.

C. Stable Functional Group Conversion

After the surface has been treated it may then be exposed to a selected gas or liquid under conditions effective to convert the active species to a stable functional group or groups 16 (FIG. 1). The time range for the conversion may be between about 1 second and about 6 minutes. Five minutes is a preferable time for conversion. The exposing step may be performed by contacting the surface with gaseous air, ammonia, or oxygen. Alternatively, the surface may be contacted with a liquid form of water, ammonium hydroxide, or hydrazine.

Following completion of the stable functional group conversion step 16, or the previous step 14, or subsequent steps 18 or 20, the wetting behavior, surface tension, and other physical properties of the resulting surface may be analyzed. Analysis methods include, but are not limited to, time-of-flight secondary ion mass spectrometry (TOF-SIMS), neutral small-angle scattering, small-angle light scattering, transmission electron microscopy, scanning electron microscopy, phase contrast microscopy, polarization microscopy, electron spectroscopy for chemical analysis (ESCA), Fourier transform infrared spectroscopy (FTIR), individual super-resolution nuclear magnetic resonance (NMR), pulsed NMR, mechanical relaxation, dielectric relaxation, X-ray photoelectron spectroscopy (XPS), DSC, DTA, TOA, fluorescent methods, spin probe methods, positron annihilation, SIMS, microscopic Raman, and the like.

D. Surface-Modifying Group Attachment

Thereafter, the exposed surface is optionally contacted with a surface-modifying group 18 under conditions effective to covalently attach the surface-modifying group to the functional group 16 or the plasma activated surface 14. Thus, the selected chemical group on the surface is the stable functional group 16 or the surface-modifying group 18 covalently attached thereto. In one embodiment the surface-modifying group 18 is a carboxylic acid group which may result in increased fibronectin and/or decreased albumin adsorption, therefore improving cell attachment and proliferation on the substrate.

Optionally, the attachment of the surface-modifying group is performed in the presence of a wetting agent to enhance the wetting of the substrate for a more uniform coating application. Useful examples of wetting agents include, but are not limited to alcohols, ethers, esters, amides, e.g. dimethylformamide (DMF), 1-butanol, n-butyl acetate, dimethyl acetamide (DMAC), and mixtures and combinations thereof.

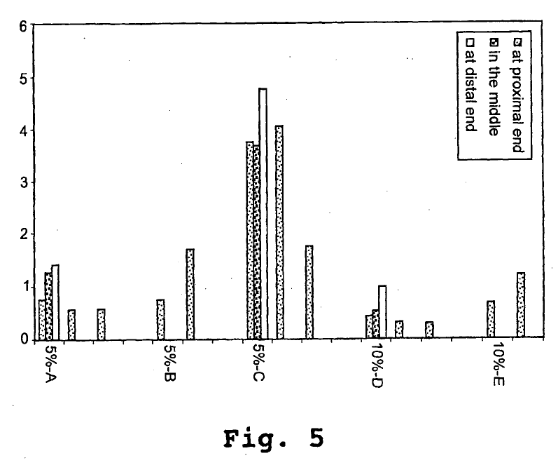

Preferable wetting agents include ethanol (EtOH) or tetrahydrofuran (THF). An exemplary method, described in Example 1, includes prewetting the substrate with 100% THF or 50:50 EtOH:NaOH solution prior to contact with an activation plasma such as chloroacetic acid/sodium hydroxide (Cl-Hac/NaOH). In this example, ePTFE was used as the substrate. As shown in FIG. 5, of the different ethanol volume percentages tested, the highest concentration of P-15 on the surface of the ePTFE was achieved at the highest excess of Cl-Hac and NaOH (5% EtOH, 1.85 M Cl-Hac, and 4.5 M NaOH). FIG. 6 shows the results of a comparison of EtOH and THF in the Cl-Hac activation process using the parameters given in Table 1. As can be seen from FIG. 6, Cl-Hac activation using THF resulted in greater than 50% higher concentrations of P-15 on the ePTFE surface. THF as a wetting agent provides the advantage of not reacting with Cl-Hac. Therefore, it is generally easier to perform the reaction under large excesses of Cl-Hac and NaOH in the presence of THF as compared to EtOH.

In some circumstances, the interaction of the stable functional group within a physiological environment or with an immobilized bioactive/biocompatible agent described below may be suboptimal. Stable functional groups may require covalent attachment of bioactive/biocompatible coatings under harsh conditions which could diminish the biological activity. Mesyl chloride, tosyl chloride, and tresyl chloride, for example, react very efficiently with less reactive groups, such as hydroxyl groups, on the substrate. In addition, they are also very good leaving groups, thus having the ability to improve the coating of the surface with sensitive or less stable bioactive/biocompatible agents under mild conditions.

In another aspect, steric hindrances between the functional group and the immobilized bioactive/biocompatible agents may limit the approach of the bioactive/biocompatible agent to the functional group. In addition, physical bulk, electrostatic repulsion, or inappropriate positioning of the bioactive/biocompatible agent or agents may also contribute to reduced efficiency of the immobilized bioactive/biocompatible agent or agents. Furthermore, a surface with many free amine groups may cause thrombogenic responses. Accordingly, it may be desirable to place one or more additional compounds as a multi-functional linker between the chemically functional groups and the bioactive/biocompatible agents to increase the space between the layer and the bioactive/biocompatible agents or reduce undesirable responses.

Suitable compounds for use as multi-functional linkers include, but are not limited to anhydrides, alcohols, acids, amines, epoxies, isocyanates, silanes, halogenated groups, and polymerizable groups. Preferably the multifunctional linker is a halogenated carboxylic acid such as chloroacetic acid, chlorobutyric acid, and chlorovaleric acid. An exemplary method of creating a compatible surface having free carboxylic groups may be achieved by reacting free amine groups with succinic anhydrides as described in U.S. Pat. No. 6,156,531, which is incorporated by reference herein. The multifunctional linker is preferably comprised of at least one molecule with 2-20 carbon atoms in the backbone. In one embodiment, the multifunctional linker is a string formed of heterofunctional molecules. Alternatively, the multifunctional linker is a string formed of alternate homofunctional molecules. It is to be understood that the functional group may itself serve as a spacer arm without necessitating the use of a separate multi-functional group compound.

E. Bioactive/Biocompatible Coating

Following coating the surface 10 with a selected chemical group 16 or 18 at a selected surface density, the surface may be further contacted with a bioactive or biocompatible agent 20 as illustrated in FIG. 1 and mentioned above. In this step, the available functional groups 16 or surface-modifying groups 18 are used to covalently or non-covalently bind the bioactive/biocompatible agent possessing desirable properties to substrate 10.

The covalent immobilization of bioactive/biocompatible agents onto substrate members according to the present invention is generally non-reversible, i.e., the bioactive/biocompatible agent is not readily released from the functional group or surface-modifying group. However, multi-functional groups capable of selectively releasing an immobilized bioactive/biocompatible agent, including therapeutic drugs, have utility in receptor/ligand interactions, molecular identification and characterization of antibody/antigen complexes, and selective purification of cell subpopulations, etc. In addition, a selectively cleavable multifunctional linker affords predictable and controlled release of bioactive/biocompatible agents from the substrate.

Thus, the invention includes in one aspect a cleavable multi-functional linker. In this embodiment, selective release of the bioactive/biocompatible agent is performed by cleaving the spacer compound under appropriate reaction conditions including, but not limited to, photon irradiation, enzymatic degradation, oxidation/reduction, or hydrolysis, for example. The selective cleavage and release of immobilized agents may be accomplished using techniques known to those skilled in the art. See for example, Horton and Swaisgood, 1987; Wong, 1991; and U.S. Pat. No. 4,745,160, which is incorporated herein by reference. Suitable compounds for use as cleavable multifunctional linkers include, but are not limited to, polyhydroxyacids, polyanhydrides, polyamino acids, tartarates, and cysteine-linkers such as Lomant's Reagent.

Bioactive/biocompatible agents may be immobilized onto the substrate using bioconjugation techniques known to those skilled in the art. See Mosbach, 1987; Hermanson, et al. 1992; and Brinkley, 1992; for example. Mild bioconjugation schemes are preferred for immobilization of bioactive/biocompatible agents in order to eliminate or minimize damage to the structure of the substrate, the functional groups, the surface-modifying groups, and/or the bioactive/biocompatible agents.

Bioactive/biocompatible agents of the present invention are typically those that are intended to enhance or alter the function or performance of a particular substrate or alter the reactions and functions of the surrounding tissues. In one embodiment, biomedical devices for use in physiological environments are substrates contemplated by the present invention. In a particularly preferred embodiment, the bioactive/biocompatible group is selected from the group consisting of cell attachment factors, growth factors, antithrombotic factors, binding receptors, ligands, enzymes, antibiotics, and nucleic acids. The use of one bioactive/biocompatible agent on a substrate is presently preferred. However, the use of two or more bioactive/biocompatible agents on a substrate is also contemplated in one embodiment of the invention.

In a related embodiment, the invention includes a first bioactive/biocompatible agent that may be released slowly, and a second bioactive/biocompatible agent that may be released faster, e.g. by physical desorption. This combination would have an advantage in different phases in the course of disease treatment, wound healing, or incorporation of an implantable device. An exemplary slow release agent is released by hydrolysis of an ester bond formed between an OH group on the bioactive agent and the COOH formed on the substrate surface.

Desirable cell attachment factors include attachment peptides, as well as (I don't want to go into discussion about what is small) active domains of large proteins or glycoproteins typically 100-1000 kilodaltons in size, which in their native state can be firmly bound to a substrate or to an adjacent cell, bind to a specific cell surface receptor, and mechanically attach a cell to the substrate or to an adjacent cell. Attachment factors bind to specific cell surface receptors, and mechanically attach cells to the substrate or to adjacent cells. Such an event typically occurs within, well defined, active domains of the attachment factors. Factors that attach cells to the substrate are also referred to as substrate adhesion molecules herein. Factors that attach cells to adjacent cells are referred to as cell-cell adhesion molecules herein. (Alberts et. al., 1994). In addition to promoting cell attachment, each type of attachment factor can promote other cell responses, including cell migration and differentiation. Suitable attachment factors for the present invention include substrate adhesion molecules such as the proteins laminin, fibronectin, collagens, vitronectin, tenascin, fibrinogen, thrombospondin, osteopontin, von Willibrand Factor, and bone sialoprotein, or active domains thereof. Other suitable attachment factors include cell-cell adhesion molecules, also referred to as cadherins, such as N-cadherin and P-cadherin.

Attachment factors useful in this invention typically comprise amino acid sequences or functional analogues thereof that possess the biological activity of a specific domain of a native attachment factor, with the attachment peptide typically being about 3 to about 20 amino acids in length. Native cell attachment factors typically have one or more domains that bind to cell surface receptors and produce the cell attachment, migration, and differentiation activities of the parent molecules. These domains consist of specific amino acid sequences, several of which have been synthesized and reported to promote the attachment, spreading and/or proliferation of cells. These domains and functional analogues of these domains are termed attachment peptides.

Exemplary attachment peptides from fibronectin include, but are not limited to, RGD or Arg Gly Asp (SEQ ID NO:2; Kleinman, et. al., 1993), REDV or Arg Glu Asp Val (SEQ ID NO:3; Hubbell, et. al., 1992), and C/H-V (WQPPRARI or Trp Gln Pro Pro Arg Ala Arg Ile) (SEQ ID NO:4; Mooradian, et. al., 1993).

Exemplary attachment peptides from laminin include, but are not limited to, YIGSR or Tyr Ile Gly Ser Arg (SEQ ID NO:5) and SIKVAV or Ser Ile Lys Val Ala Val (SEQ ID NO:6) (Kleinman, et al., 1993) and F-9 (RYVVLPRPVCFEKGMNYTVR or Arg Tyr Val Leu Pro Arg Pro Val Cys Phe Glu Lys Gly Met Asn Tyr Thr Val Arg) (SEQ ID NO:7; Charonis, et al., 1988).

Exemplary attachment peptides from collagen include, but are not limited to, HEP-III (GEFYFDLRLKGDK or Gly Glu Phe Tyr Phe Asp Leu Arg Leu Lys Gly Asp Lys) (SEQ ID NO:8; Koliakos, et al., 1989) and P-15 (GTPGPQGIAGQRGVV; SEQ ID NO: 1). Desirably, attachment peptides used in this invention have between about 3 and about 30 amino acid residues in their amino acid sequences. Preferably, attachment peptides have not more than about 15 amino acid residues in their amino acid sequences. In one embodiment, attachment peptides have exactly 15 amino acid residues in the amino acid sequences.

An embodiment of the present invention involves synthetic compositions that have a biological activity functionally comparable to that of all or some portion of P-15. By “functionally comparable,” is meant that the shape, size, and flexibility of a compound is such that the biological activity of the compound is similar to the P-15 region, or a portion thereof. Biological activities of the peptide may be assessed by different tests including inhibition of collagen synthesis, inhibition of collagen binding, and inhibition of cell migration on a collagen gel in the presence of the peptide in solution. Of particular interest to the present invention is the property of enhanced cell binding. Useful compounds could be selected on the basis of similar spatial and electronic properties as compared to P-15 or a portion thereof. These compounds typically will be small molecules of 50 or fewer amino acids or in the molecular weight range of up to about 2,500 daltons, more typically up to about 1000 daltons. Inventive compounds of the invention include synthetic peptides; however, nonpeptides mimicking the necessary conformation for recognition and docking of collagen binding species are also contemplated as within the scope of this invention. For example, cyclic peptides on other compounds in which the necessary conformation is stabilized by nonpeptides (e.g., thioesters) is one means of accomplishing the invention.

The central portion, forming a core sequence, of the P-15 region has been identified as having collagen-like activity. Thus, bioactive/biocompatible agents of this invention may contain the sequence Gly-Ile-Ala-Gly (SEQ ID NO:9). The two glycine residues flanking the fold, or hinge, formed by -Ile-Ala- are hydrogen bonded at physiologic conditions and thus stabilize the [beta]-fold. Because the stabilizing hydrogen bond between glycines is easily hydrolyzed, two additional residues flanking this sequence can markedly improve the cell binding activity by further stabilizing the bend conformation. An exemplary bioactive/biocompatible agent with advantageous properties contemplated by the present invention, having glutamine at each end (Gln-Gly-Ile-Ala-Gly-Gln; SEQ ID NO: 10) is described in U.S. Pat. No. 6,268,348, issued Jul. 31, 2001, which is incorporated by reference in its entirety herein.

Example 2 illustrates that substrates treated in accordance with the method of the invention have the ability to provide enhanced endothelial cell growth in vitro. The example characterized the P-15 surface treatment on ePTFE graft material, and measured its biological activity on the adhesion, migration and proliferation of endothelial cells in vitro. Also shown is the level of P-15 treatment degradation after simulated aging. The results show that this treatment method, characterized by the covalent attachment of a cell-adhesion peptide, was shown to be clean and stable. The surface treatment on ePTFE grafts promoted the migration and proliferation of healthy endothelial cells. Thus, the coating method of the invention may improve the speed and quality of endothelialization, and finds utility in reducing device failure caused by thrombosis and restinosis of vascular devices.

Exemplary antiproliferative agents include angiopeptin (a somatostatin analog from Ibsen), angiotensin converting enzyme inhibitors such as CAPTOPRIL (available from Squibb), CILAZAPRIL (available from Hoffman-LaRoche), or LISINOPRIL (available from Merck); calcium channel blockers (such as Nifedipine), colchicine, fibroblast growth factor (FGF) antagonists, fish oil (omega 3-fatty acid), histamine antagonist, LOVASTATIN (an inhibitor of HMG-CoA reductase, a cholesterol lowering drug from Merck), monoclonal antibodies (such as PDGF receptors), nitroprusside, phosphodiesterase inhibitors, prostaglandin inhibitor (available form Glazo), Seramin (a PDGF antagonist), serotonin blockers, steroids, thioprotease inhibitors, triazolopyrimidine (a PDGF antagonist), and nitric oxide. Of particular interest is rapamycin, which is capable of inhibiting the inflammatory response following graft, stent, or stent-graft implantation. Also preferred is TAXOL. Additional exemplary bioactive/biocompatible agents may be found in U.S. Pat. No. 6,299,604 which is incorporated herein by reference.

Other desirable bioactive/bioactive agents present in the invention include growth factors, such as fibroblastic growth factors, epidermal growth factor, platelet-derived growth factors, transforming growth factors, vascular endothelial growth factor, bone morphogenic proteins and other bone growth factors, neural growth factors, and the like.

Yet other desirable bioactive/biocompatible agents of the present invention include antithrombotic agents that inhibit thrombus formation or accumulation on blood contacting devices. Desirable antithrombotic agents include heparin and hirudin, which inhibit clotting cascade proteins such as thrombin, as well as lysin. Other desirable antithrombotic agents include prostaglandins such as PGI2, PGE1, and PGD2, which inhibit platelet adhesion and activation. Still other desirable antithrombotic agents include fibrinolytic enzymes such as streptokinase, urokinase, and plasminogen activator, which degrade fibrin clots. Another desirable bioactive/biocompatible agent consists of lysin, which binds specifically to plasminogen, which in turn degrades fibrin clots.

Other desirable bioactive/biocompatible agents present in the invention include binding receptors, such as antibodies and antigens. Antibodies present on a substrate can bind to and remove specific antigens from aqueous media that comes into contact with the immobilized antibodies. Similarly, antigens present on a substrate can bind to and remove specific antibodies from aqueous media that comes into contact with the immobilized antigens.

Other desirable bioactive/biocompatible agents consist of receptors and their corresponding ligands. For example, avidin and streptavidin bind specifically to biotin, with avidin and streptavidin being receptors and biotin being a ligand. Similarly, fibroblastic growth factors and vascular endothelial growth factor bind with high affinity to heparin, and transforming growth factor beta and certain bone morphogenic proteins bind to type IV collagen. Also included are immunoglobulin specific binding proteins derived from bacterial sources, such as protein A and protein G, and synthetic analogues thereof.

Yet other desirable bioactive/biocompatible agents of the present invention include enzymes that can bind to and catalyze specific changes in substrate molecules present in aqueous media that comes into contact with the immobilized enzymes. Other desirable bioactive/biocompatible agents consist of nucleic acid sequences (e.g., DNA, RNA, and cDNA), which selectively bind complimentary nucleic acid sequences. Substrate surfaces coated with specific nucleic acid sequences are used in diagnostic assays to identify the presence of complimentary nucleic acid sequences in test samples.

Still other desirable bioactive/biocompatible agents of the present invention include antibiotics that inhibit microbial growth on substrate surfaces. Certain desirable antibiotics may inhibit microbial growth by binding to specific components on bacteria. A particularly desirable class of antibiotics are the antibiotic peptides which seem to inhibit microbial growth by altering the permeability of the plasma membrane via mechanisms which, at least in part, may not involve specific complimentary ligand-receptor binding (Zazloff, 1992).

In one embodiment, the bioactive or biocompatible agent is bound to the surface at a density of 0.01 to 1000 nmol/cm². Preferably, the bioactive or biocompatible agent is bound to the surface at a density of 0.5 to 30 nmol/cm². More preferably, the bioactive or biocompatible agent is bound to the surface at a density of 2 to 20 nmol/cm².

Chemical/biological testing such as AAA (amino acid analysis), GC/MS (gas chromatography/mass spectrometry), accelerated aging, in vitro cell cultures followed by SEM (scanning electron microscopy), and in vivo testing may be used to evaluate the coatings of the present invention as described in Examples 2 and 3 below. Biocompatibility tests known to those of skill in the art, such as cytotoxicity, in vitro hemolysis, muscle implantation, and genotoxicity e.g. bacterial reverse mutation, saline and DMSO extracts, chromosomal aberration, and mouse bone marrow micronucleus may also be used.

### III. Exemplary Devices

In another aspect, the invention includes a substrate having a coating prepared in accordance with the method above. The substrate is preferably part of a medical implant or medical device that has at least one surface, or a portion thereof, which is to be treated to achieve a desirable biological effect on that surface. Exemplary devices include stents, grafts, catheters, catheter guidewires, wound drainage devices, dressings, intraocular lenses, pacemakers, and cardiac valves. The substrates/devices may be implanted into a patient in need thereof according to the well-known procedures routinely used in the field of biomedical implants. Exemplary devices include unsupported vascular grafts and supported stent grafts.

From the foregoing it can be seen how various objects and features of the substrate coating invention are met.

### IV. Examples

The following examples further illustrate the invention described herein and are in no way intended to limit the scope of the invention.

### Example 1

### Chloroacetic Acid Activation Using Wetting Agents

This example characterized the plasma activation of an ePTFE surface with Cl-Hac using EtOH as a reacting wetting agent or using THF as a non-reacting wetting agent. The inside surface of an ePTFE graft having an internal diameter of 3 mm and 0.003″ wall thickness was treated as described. Activation was performed with Cl-Hac/NaOH in the presence of a wetting agent. Grafts were prewet with 100% THF or 50:50 EtOH:NaOH solution before contact with Cl-Hac/NaOH. Grafts were then treated with P-15 peptide/EDC in a DMSO/water solution. Grafts were then rinsed with water, and then with 10% EtOH in water and dried. The surface treated grafts were then analyzed in 2 cm pieces by amino acid analysis and Outgassing GC/MS.

FIG. 5 shows the concentration of P-15 resulting from the use of EtOH as a wetting agent. 5%-A refers to 5% EtOH+1.35 M Cl-Hac+3 M NaOH. 5%-B refers to 5% EtOH+1.85 M Cl-Hac+4 M NaOH. 5%-C refers to 5% EtOH+1.85 M Cl-Hac+4.5 M NaOH. 10%-D refers to 10% EtOH+2.2 M Cl-Hac+5 M NaOH. 10%-E refers to 10% EtOH+2.4 M Cl-Hac+5.4 M NaOH.

FIG. 6 shows a comparison of EtOH and THF in the Cl-Hac activation process, and the resulting P-15 concentration. Cl-Hac activation using 15% THF or 5% EtOH were compared. Table 1 shows the parameters of the three plasma settings used to treat the grafts.

TABLE 1
Plasma settings for treating grafts

| | MAX | MED | MIN |
|---|---|---|---|
| Voltage (V) | 20 | 16 | 12 |
| PTFE Length (cm) | 20 | 14 | 8 |
| Flow (scfh) | 30 | 20 | 10 |
| Plasma time (s) | 55 | 35 | 15 |

### Example 2

### Enhanced Endothelial Growth In Vitro on ePTFE Surface Treated with P-15

A. Materials and Methods

1. Peptide Coating

GLP-grade P-15 peptide (SEQ ID NO: 1) was custom-ordered from Advanced ChemTech (Louisville, Ky.) and stored at 4° C. prior to the coating processes. Small diameter (3 mm) expanded PTFE (ePTFE) grafts were plasma activated and surface-modified according to Example 3, and covalently coated with the P-15 peptide as described in U.S. Pat. No. 6,159,531. All reactions were carried out in aqueous solutions. Small amounts of dimethyl sulfoxide (DMSO) or ethanol (EtOH) were added to increase the efficiency of chemical reactions and rinsing processes. After final rinses in aqueous solutions and drying with nitrogen gas, treated ePTFE grafts were stored in clean fluoroware containers.

2. Amino Acid Analysis (AAA)

The peptide on the surface of ePTFE grafts was quantified by amino acid analysis (AAA). In this method, peptides and proteins were separated from the grafts and broken down completely into single amino acids by hydrolysis in 6N HCl at 110° C. for 22-24 hours. The amounts of individual amino acids were quantified by ion-exchange chromatography. The total quantity of P-15, as well as the purity of the peptide (matching actual with predicted amino acid ratios) were calculated.

In some cases prior to AAA, grafts were rinsed three times ultrasonically in 0.1% sodium dodecyl sulfate (SDS) prior to the amino acid analysis to remove physically adsorbed peptides or peptide segments.

3. Outgassing Gas Chromatography/Mass Spectroscopy (GC/MS)

Residual levels of volatile solvents such as THF or ethanol were determined by Headspace analysis. Samples were sealed in a quartz tube and outgassed at 85° C. for 2 hours. The outgassed materials were condensated in a cool −10° C. capillary prior to entering a GC column. Standardization was performed utilizing pure solvents used in the coating processes.

4. Non-Purgable Total Organic Carbons (NPOC)

Residues of non-volatile solvents and chemicals, such as DMSO or carbodiimide, were determined by a dynamic extracting with water at 37° C. for 3 days. Organic carbon was measured subsequently using a UV-Persulfate TOC Analyzer.

5. Accelerated Aging Test

Treated grafts were ethylene oxide (EtO) sterilized and accelerated aging tests were performed at 55° C. at a range of humidity to stimulate 6 months and 2 years storage at room temperature.

6. Measurement of Biological Activity Using In Vitro Endothelial Cell Cultures

2-cm long ePTFE grafts, treated on the outside with P-15 peptide (P-15), and untreated (control) grafts, were supported in the inside with a stainless steel frame. Assembled grafts were sterilized using EtO.

The wells of tissue-culture multi-well plates were layered with sterile agarose gel. Sterile grafts were positioned vertically in these wells. A suspension of transformed human umbilical vein endothelial cells (ECV304) in Dulbecco's Modified Eagle's Medium and 10% fetal calf serum were seeded at the bottom of grafts. After an initial adhesion for 3 hours in the incubator, the medium was added to the top of the well to allow cell migration to the top. After 6 or 13 days, grafts were taken out and rinsed carefully with sterile phosphate buffer solution (PBS). The samples were then dried and prepared for SEM analysis.

The migration and the morphology of endothelial cells on grafts were evaluated qualitatively using SEM. The proliferation of endothelial cells on P-15 treated and control grafts was assessed quantitatively by the number of cells recovered after trypsinization.

B. Results and Discussion

1. Characterization of the Peptide Treatment

The original amino acid analysis data for grafts prior to and after the covalent binding with P-15 peptide were shown in Table 2. Nleu was used as an internal standard for the injection of amino acid mixtures into ion exchange column. It is evident that plasma treatment and other activation processes lead to a relatively clean substrate. Amino acids recovered from P-15 peptide treated grafts originated mostly from the peptide itself.

Table 3 showed that the composition of the hydrolyzed peptide coating corresponded well to the composition of free P-15 peptide. The matching scores, based on the difference between the recovered composition and the theoretical composition of individual amino acids present in P-15 peptide, were similar for free P-15 peptide powder and P-15 coated on the graft.

The total peptide concentrations on the surface and the matching scores remained in the same range for freshly coated grafts before and after surfactant rinses, as well as for grafts subjected to the aging tests equivalent to six months and two years. This indicates that covalently bound P-15 peptide is stable on the surface of ePTFE grafts over an extended period of time.

2. Residual Solvents:

P-15 treated ePTFE grafts dried by nitrogen gas and air contained 0.2 ppm ethanol and 0.2 ppm DMSO as shown by outgassing GC/MS analysis. The detection limit for each solvent in the outgassing GC/MS analysis is 0.1 ppm. Ethanol traces in blood after an alcohol drink is common, while small amounts of DMSO are used to protect in-vitro cells from freezing cycle. 0.2 ppm levels of these two solvents—ethanol and DMSO—appear acceptable here. The only possible harmful solvent, THF, could not be detected at all.

Vigorous shaking of P-15 treated grafts in water at 37° C. for 3 days could only extract 0.006 mg non-purgable organic carbon (NPOC), compared to 0.004 mg for a blank sample. These numerous characterizations of the P-15 peptide treatment on ePTFE grafts show that P-15 peptide can be attached stably and cleanly to the surface of ePTFE.

TABLE 2
Amino Acid Analysis Data for Control
and P-15 peptide treated ePTFE

| | |
|---|---|
| Concentration | |
| (pmol/injection) | Ratio |

| Amino Acid | Control | P-15 | P-15 | Theoretical |
|---|---|---|---|---|
| Asx | 20.7 | 62.7 | 0.02 | |
| Thr | | 2690.2 | 0.92 | 1 |
| Ser | 26.1 | 113.1 | 0.04 | |
| Glu | 33.4 | 6745.5 | 2.31 | 2 |
| Pro | | 6299 | 2.15 | 2 |
| Gly | 49.2 | 13394.6 | 4.58 | 5 |
| Ala | | 3166.8 | 1.08 | 1 |
| Cys | | | 0.00 | |
| Val | | 5065 | 1.73 | 2 |
| Met | | | 0.00 | |
| Ile | 10.4 | 3020.9 | 1.03 | 1 |
| Leu | 24.1 | 64.2 | 0.02 | |
| nleu (standard) | 373.7 | 371.2 | 0.13 | |
| Tyr | | 24.5 | 0.01 | |
| Phe | | 22.6 | 0.01 | |
| His | | 20.8 | 0.01 | |
| Lys | | 31.7 | 0.01 | |
| NH3 | 558.4 | 3013.4 | 1.03 | |
| Arg | | 2816.3 | 0.96 | 1 |

| | |
|---|---|
| Matching Score | 1.38 |

TABLE 3
Summary of the Amino Acid Analysis for
P-15 peptide treated ePTFE grafts

| | Avg. P15 conc. | Avg. matching |
|---|---|---|
| Sample | (nmol/cm²) | score |
| free peptide | n/a | 1.00 |
| freshly coated (no SDS rinse) | 8.30 | 1.01 |
| freshly coated (w/SDS rinse) | 8.13 | 0.87 |
| 6-month aging (w/SDS rinse) | 7.04 | 0.94 |
| 2-year aging (w/SDS rinse) | 9.44 | 0.96 |

3. Biological Activity of the P-15 Peptide Coating Using In-Vitro Endothelial Cell Cultures

A common method to test the interactions between biomaterials and cells involves seeding cells directly on flat substrate surfaces and observing cell adhesion and proliferation. However, the porous and thin-wall ePTFE graft material was very soft and difficult to keep flat. Immediately after seeding, cells often remained concentrated on deeper folds and dents. Cell adhesion in this case was more a result of the substrate topography rather than the interactions between cells and the substrate. To avoid this effect of the substrate topography, we seeded cells at the bottom of vertically positioned ePTFE grafts and let them migrate vertically upwards. In addition, the migration along ePTFE grafts corresponded more closely to the migration of endothelial cells from the two ends of implanted intravascular grafts.

SEM analysis of grafts after 6 days and 13 days in culture with endothelial cells revealed significant differences in cell distribution and morphology. After 6 days, endothelial cells stayed mainly at the bottom part of the control ePTFE while they migrated almost to the top part of the P-15 treated ePTFE. Endothelial cells on control ePTFE looked more rounded. In contrast, cells looked flatter on P-15 treated ePTFE. This smoother cell morphology is characteristic of healthier cells.

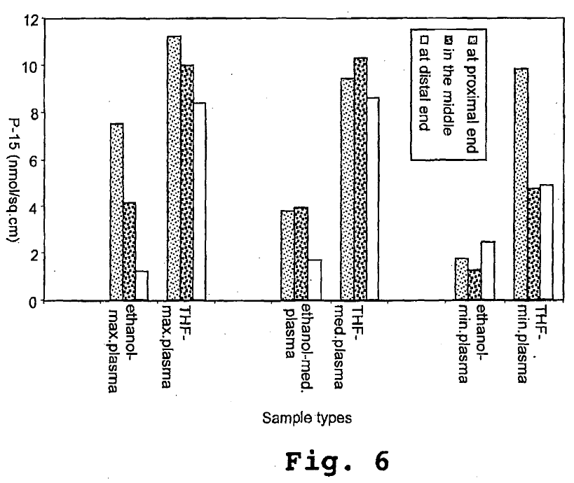

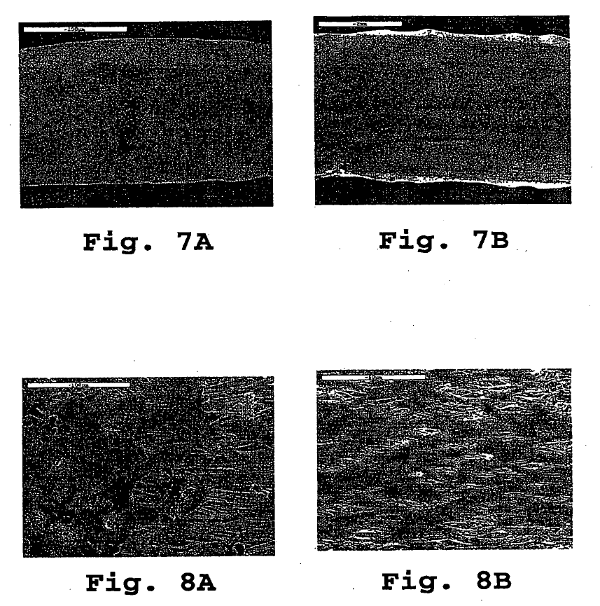

After 13 days in culture, the difference between control and P-15 treated became more significant. At low magnifications (FIGS. 7A and 7B), dark areas indicating dense endothelial cell population were larger on the P-15 treated graft (FIG. 7B) than on controls (FIG. 7A). Small dark spots indicating migrating cells had spread further from the dense seeding lines on the P-15 treated graft. The morphology of individual cells could be observed much better at higher magnifications and at the edge of migration (FIGS. 8A and 8B). Cells on the control graft (FIG. 8A) were typically rounded, while all the cells on the P-15 treated ePTFE (FIG. 8B) were flat and provided greater coverage of the porous surface of the ePTFE.

After 15 days in culture, cells grown on grafts were removed by trypsinization and counted. There were about 5 times more cells on the P-±5 treated grafts than on the controls. The distribution, morphology and number of endothelial cells in-vitro suggested that P-15 treatment favored the formation of endothelial lining on ePTFE grafts.

### Example 3

### Substrate Treatment

A. Plasma Treatment

A graft (3 mm ID; 0.003″ thick; 50-60 μm IND) was attached with fittings to the plasma nozzle under argon gas flow (no power). Argon gas is allowed to flow at 28-32 scfh for 20-30 seconds and then stopped. Allow argon gas and plasma to flow with power (11.5-14.5V; 0.35-0.65 A) on for 30 seconds. Remove graft from the plasma and put on clean surface. Allow at least 5 minutes to elapse prior to the next step.

B. Chloroacetic Acid Activation

Place graft in a 50 ml polypropylene tube. Pipet 5 ml THF into the centrifuge tube and then add 40 ml freshly prepared chloroacetic acid (IM C-Hac in 3M NaOH). Shake by hand to mix the final solution, then place on a platform shaker for 16 to 24 hours at room temperature. Rinse graft thoroughly to remove all chemical residues and proceed to the peptide coating step using carbodimide chemistry as described in U.S. Pat. No. 6,159,531, which was incorporated by reference above.

TABLE 4
Sequence Provided in Support of the Invention.

| | |
|---|---|
| | SEQ. |
| Description | ID |

| | |
|---|---|
| P15 | 1 |
| GTPGPQGIAGQRGVV | |
| RGD | 2 |
| REDV | 3 |
| C/H-V | 4 |
| WQPPRARI | |
| YIGSR | 5 |
| SIKVAV | 6 |
| F-9 | 7 |
| RYVVLPRPVCFEKGMNYTVR | |
| HEP-III | 8 |
| GEFYFDLRLKGDK | |
| Gly-Ile-Ala-Gly | 9 |
| Gln-Gly-Ile-Ala-Gly-Gln, | 10 |

Although the invention has been described with respect to particular embodiments, it will be apparent to those skilled in the art that various changes and modifications can be made without departing from the invention.

1. A method of forming on the surface of a substrate a coating having a selected surface density of a selected chemical group, said method comprising treating the surface with a plasma to form one or more active species on the surface until a desired surface density of the active species is formed; exposing the treated surface under conditions effective to convert the active species to a stable functional group; and contacting the exposed surface to a surface-modifying group under conditions effective to attach the surface-modifying group to the stable functional group, the improvement comprising:
performing the treating with the plasma at or near atmospheric pressure;
performing the contacting to attach the surface modifying group to the stable group with of a surface modifying group, where the surface modifying group is a carboxylic acid group; and
further contacting the exposed surface with material selected from an antithrombotic agent, a cell attachment factor, a receptor, a ligand, a growth factor, an antibiotic, and an enzyme.

2-6. (canceled)

7. The method of claim 1, wherein the plasma is formed of a carrier gas and less than ten percent of a gas or vapor selected from oxygen, water, ammonia, ammonium hydroxide, an organic amine, an alcohol, an aldehyde, a carboxylic acid and an ester.

8-10. (canceled)

11. The method of claim 1, wherein the antithrombotic agent is selected from heparin, hirudin, lysine, prostaglandin, streptokinase, urokinase, and plasminogen activator.

12. The method of claim 1, wherein the cell attachment factor is selected a surface adhesion molecule and a cell-cell adhesion molecule.

13. The method of claim 12, wherein the surface adhesion molecule is selected from laminin, fibronectin, collagen, vitronectin, tenascin, fibrinogen, thrombospondin, osteopontin, von Willibrand Factor, and bone sialoprotein, and active domains thereof.

14. The method of claim 12, wherein the cell-cell adhesion molecule is P15.

15. The method of claim 12, wherein the cell-cell adhesion molecule is selected from N-cadherin and P-cadherin and active domains thereof.

16. The method of claim 1, wherein the growth factor is selected from a fibroblastic growth factor, an epidermal growth factor, a platelet-derived growth factor, a transforming growth factor, a vascular endothelial growth factor, a bone morphogenic protein, and a neural growth factor.

17. The method of claim 1, wherein said the ligand or receptor is selected from an antibody, an antigen, avidin, streptavidin, biotin, heparin, type IV collagen, protein A, and protein G.

18. The method of claim 1, wherein the antibiotic is an antibiotic peptide

19-26. (canceled)

27. The method of claim 1, wherein the carboxylic acid group is halogenated.

28. The method of claim 27, wherein the halogenated carboxylic acid group is selected from chloroacetic acid, chlorobutyric acid, and chlorovaleric acid.

29-32. (canceled)

33. The method of claim 1, wherein the plasma is formed of a carrier gas alone.

34. The method of claim 33, wherein the carrier gas is selected from nitrogen, carbon dioxide and noble gasses.

35. The method of claim 34, wherein the carrier gas includes argon.
